(19) United States
(12) Patent Application Publication
(10) Pub. No.: US 2007/0081358 A1
(43) Pub. Date: Apr. 12, 2007

(54) Medical Illumination Device with a Base

(76) Inventors: Todd Shea, Victoria, MN (US); Kevin Arnal, Excelsior, MN (US)

Correspondence Address: MAEYAERT & MAEYAERT, 2091 COUNTY ROAD D EAST, SUITE C-100, MAPLEWOOD, MN, 55109, US

(21) Appl. No.: 11/163,188

(22) Filed: Oct. 8, 2005

(51) Int. Cl. F21S 4/00; A61B 1/06

(52) U.S. Cl. 362/572

(57) ABSTRACT

A medical illumination device including a base, a light housing and a flexible body. The base includes a power source and an attachment component. The attachment component further includes a first portion and a second portion, the first portion attachable to an attachment point and the second portion repositionable relative to the first portion without detaching the first portion from the attachment point. The flexible body has a self-sustaining shape with at least one bendable shaft positioned inside the flexible body. The base provides stability for repositioning of the flexible body. The base has sufficient surface area contacting the attachment point, sufficient base weight, and sufficient holding strength to effectively provide stability for repositioning of the flexible body. The base comprises at least 50% of the weight of the device and has a surface area of at least 1.0 square inch.

The present invention relates to the field of medical, surgical, and dental illumination devices. More particularly, this invention relates to an illumination device that includes a stable base and is also self-contained, compact, flexible, portable, high intensity, and low heat producing and readily contained in a sterile package. The present invention also overcomes problems of handless stability as well as sterility in various medical environments, especially in emergency response situations where other sterile light sources are not readily available.

### BACKGROUND OF INVENTION

A significant part of any medical procedure, therapy, or assessment is an adequate light source to provide the user with a highly perceptible view of the area of interest. Many sources of illumination are currently used to provide diffuse lighting, but supply inadequate lighting of specific areas of interest. The areas of interest may include small structures, deep spaces, or within small incisions.

Conventional lighting systems in the operative theater consist of one or many large overhead lighting units. These systems work well for general surgical field illumination, but are less efficient at illuminating deep spaces or areas within small incisions. Due to the distance between the light source and the specific area of interest within the surgical field, the area of interest may not be easily illuminated. The adjustment of the orientation and focus of the light may be less than ideal due patient positioning, surgical team member location, or instrumentation used during the procedure. The large size of such lights cause excessive heat generation that may adversely effect the surgical environment. Patient tissue is directly affected by any increase of temperature and overheating may lead to eventual tissue injury. In addition, contamination of surgical team members is not uncommon with the use of the overhead lighting systems due to their location and the cumbersome nature of the large lights.

It is important to maintain a clean and sterile environment to eliminate the risk of infection to the patient or risk of infection to the surgical team. Furthermore, to provide the best surgical outcome for the patient, a procedure which can be completed efficiently with short operative times and a minimal drying of the tissue is important.

Head lamps have been utilized to allow for improved illumination of deep spaces or within small incisions. Such apparatus is cumbersome, uncomfortable, and restricts surgical team motion during the procedure. Head lamps are also limited in ease of adjustment and orientation. For example, as the head of the user moves, so does the orientation of the light, providing for inconsistent illumination. In addition, due to the non-sterile nature of the headlamp, adjustment of the headlamp requires a non-sterile member of the surgical team to provide for initial orientation of the light and any subsequent adjustments during the procedure.

Most head lamps use fiber optic light sources. Often users of the head lamps feel tethered by the fiber optic connection. The attachment of the head lamp to the light source is very restrictive to the user's motion and requires help from the non-sterile members of the surgical team for connecting and moving of the light source. Due to the non-sterile nature of the head lamp, contamination must be a concern when using such a lighting method.

Many other devices for illumination have been introduced, but are not readily used due to their inherent short comings. Most surgical lighting methods that are capable of close proximity to the area of interest are powered by fiber optic sources. Some of these may include bendable or flexible properties. However, this type of device is limited in its mobility and function due to the light source itself. With such a device being attached to an extrinsic illumination source it is functionally limited due to the “tethering effect”, lack of mobility, and risk of contamination of the sterile field.

Other devices provide self contained illumination, but have other short comings. For example, such devices may have limitations due to their lack of a stable mounting or base, potential for excessive heat production, and inability to easily manipulate the light beam. Such self contained illumination devices may be integral with a surgical instrument, separately attached directly to a surgical tool, or hand held. However, such self contained illumination devices lack consistency of illumination due to the inability of the person holding the implements to remain stable over long periods of time.

Another short coming is that prior devices require physically redirecting the entire light source and are thereby limited in quickly redirecting and maintaining the light beam.

Lastly, prior devices are not easily sterilizable or not provided with a portable sterile package, which limits the devices portability and usability, especially by first responders in field use.

### SUMMARY OF THE INVENTION

One aspect of the invention includes a self contained, compact, selectively mountable, easily positionable, portable, high intensity, low heat producing light source.

This aspect of the invention overcomes the problem of a non-sterile light source that can be used in close proximity to the area of interest due to its self contained, compact, and sterile properties. The selective mounting base allows the device to be mounted on a number of surfaces including operative drapes, patient's clothes (for emergency field use) and other convenient areas to illuminate the patient site. With the removable mounting ability, the device may be connected relative to the patient so its orientation will not change with instrumentation or operative staff motion. The easily directable body of the device allows for relatively instantaneous unlimited orientation adjustment of the light beam direction without relocating the mounting base.

Because the intensity of light is related by the inverse square of the distance from the light, it often takes a very intense, high-powered light located overhead of the medical personnel to provide enough illumination. Such a high intensity light can create significant heat, which can dry out exposed patient tissue and promote increased cell death. Having a low heat producing light source so close to the focus of interest requires less intense light and less chance of tissue damage. With the light source in such close proximity to the focal area of interest, blocking the light by surgical team members or instrumentation is less likely, thus promoting a more efficient and faster surgery.

In the case of emergency medical procedures in the field or diagnostic or therapeutic procedures which may be conducted when there is not a light source available, the portability of this device is very desirable. This aspect of the invention would provide a beam of light directly on the patient site in settings that are currently not accessible by previously introduced illumination devices.

One aspect of the invention provides a medical illumination device including a base, a light housing, and a flexible body. The base contains a power source, and includes an attachment component that is attachable to an attachment point. The light housing encloses a light source. The flexible body is attached to the base and the light housing. The light source is electrically connected to the power source via conductors enclosed in the flexible body. The flexible body has a self-sustaining shape. The base provides stability for repositioning of the flexible body.

Another aspect of the invention provides that the base may have sufficient surface area contacting the attachment point to effectively provide stability for repositioning of the flexible body. The base may have a surface area of at least 1.0 square inch. The base may comprise at least 50% of the weight of the device. The base may have sufficient surface area contacting the attachment point, sufficient base weight, and sufficient holding strength to effectively provide stability for repositioning of the flexible body. The flexible body may include at least one bendable shaft positioned inside the flexible body. The light housing may include a lens for adjusting of a light beam and a filter for improving visibility. The flexible body may be sealably attached to the base and the light housing may sealably enclose the light source. The device may include a second light housing enclosing a second light source and a second flexible body attaching the second light housing to the base. The attachment component may include a first portion and a second portion, wherein the first portion is attachable to the attachment point and the second portion is repositionable relative to the first portion without detaching the first portion from the attachment point.

One aspect of the invention provides a medical illumination device including a base, a light housing, and a flexible body. The base contains a power source, and includes an attachment component. The light housing encloses a light source. The flexible body is attached to the base and the light housing. The light source electrically connects the power source via conductors enclosed in the flexible body. The flexible body has a self-sustaining shape. The attachment component includes a first portion and a second portion. The first portion is attachable to an attachment point and the second portion is repositionable relative to the first portion without detaching the first portion from the attachment point.

Another aspect of the invention provides an attachment component that may include a hook and loop pad. The flexible body may include at least one bendable shaft positioned inside the flexible body. The light housing may include a lens for adjusting of a light beam and a filter for improving visibility. The flexible body may be sealably attached to the base and the light housing may sealably enclose the light source. The base may have sufficient surface area contacting the attachment point, sufficient base weight, and sufficient holding strength to effectively provide stability for repositioning of the flexible body. The base may comprise at least 50% of the weight of the device and may have a surface area of at least 1.0 square inch. The device may include a second light housing enclosing a second light source and a second flexible body attaching the second light housing to the base.

One aspect of the invention provides a medical illumination device including a base, a light housing, and a flexible body. The base contains a power source including an attachment component. The attachment component includes a first portion and a second portion. The first portion is attachable to an attachment point and the second portion is repositionable relative to the first portion without detaching the first portion from the attachment point. The light housing encloses a light source. The flexible body attaches the base to the light housing. The light source is electrically connected to the power source via conductors enclosed in the flexible body. The flexible body has a self-sustaining shape. The flexible body includes at least one bendable shaft positioned inside the flexible body. The base provides stability for repositioning of the flexible body and the base has sufficient surface area contacting the attachment point, sufficient base weight, and sufficient holding strength to effectively provide stability for repositioning of the flexible body. The base comprises at least 60% of the weight of the device and the base has a surface area of at least 1.0 square inch.

Another aspect of the invention may further provide a second light housing enclosing a second light source and a second flexible body attaching the second light housing to the base. The light housing may further include a lens and a filter. The flexible body may be sealably attached to the base and the light housing may sealably enclose the light source.

One aspect of the invention provides a medical illumination device system having a base, a light housing, a flexible body, and a sterile package. The base contains a power source and includes an attachment component. The light housing encloses a light source. The flexible body is attached to the base and the light housing. The light source is electrically connected to the power source via conductors enclosed in the flexible body. The flexible body has a self-sustaining shape. The sterile package has a sterile barrier and encloses the base, the light housing, and the flexible body within the sterile barrier.

Another aspect of the invention may include an attachment component that includes an adhesive that is exposable upon removal of the attachment component from the sterile package. The attachment component may include a coversheet. The coversheet may be attached to the sterile package so that removal of the coversheet from the attachment component facilitates removal of the attachment component from the sterile package. The coversheet may be integral with the sterile package. The attachment component may include a first portion and a second portion. The first portion may be attachable to an attachment point. The second portion may be repositionable relative to the first portion without detaching the first portion from the attachment point. The first portion and the second portion may be separately removable from the sterile package. The base may provide stability for repositioning of the flexible body and the base may have sufficient surface area contacting the attachment point to effectively provide stability for repositioning of the flexible body. The base may have sufficient surface area contacting the attachment point, sufficient base weight, and sufficient holding strength to effectively provide stability for repositioning of the flexible body. The base may comprise at least 50% of the weight of the device and have a surface area of at least 1.0 square inch. The flexible body may include at least one bendable shaft positioned inside the flexible body. The light housing may also include a lens and a filter. The flexible body may be sealably attached to the base and the light housing may sealably enclose the light source. The device may include a second light housing enclosing a second light source and a second flexible body attaching the second light housing to the base.

One aspect of the invention provides a medical illumination device system having a sterile package, a base, a light housing, and a flexible body. The sterile package has a sterile barrier for enclosing the device within the sterile barrier. The base contains a power source and an attachment component. The attachment component includes a first portion and a second portion. The first portion is attachable to an attachment point and the second portion is repositionable relative to the first portion without detaching the first portion from the attachment point. The light housing encloses a light source. The flexible body attaches the base and the light housing. The light source electrically connects the power source via conductors enclosed in the flexible body. The flexible body has a self-sustaining shape and includes at least one bendable shaft positioned inside the flexible body. The base provides stability for repositioning of the flexible body and has sufficient surface area contacting the attachment point, sufficient base weight, and sufficient holding strength to effectively provide stability for repositioning of the flexible body. The base comprises at least 60% of the weight of the device and has a surface area of at least 1.0square inch.

Another aspect of the invention may provide a second light housing enclosing a second light source and a second flexible body that attaches the second light housing to the base. The light housing may include a lens and a filter. The flexible body may be sealably attached to the base and the light housing may sealably enclose the light source.

One aspect of the invention provides a medical illumination device having a base containing a power means, a lighting means, and a flexible body means. The base further includes an attachment means for securely and stably attaching the base. The flexible body means provides for a flexible, bendable, repositionable, self-sustaining shaped body attached between the base and the lighting means. The lighting means is electrically connected to the power means via conductors enclosed in the flexible body means.

The device may also include a sterile packaging means for providing a sterile barrier enclosing the device. The device may also include repositioning means so that a first portion of the attachment means remains attached to an attachment point, and a second portion of the attachment means is repositioned relative to the first portion without detaching the first portion from the attachment point.

### BRIEF DESCRIPTION OF THE DRAWINGS

Reference will hereinafter be made to the following drawings in which like reference numerals throughout the various FIGS. denote like structural elements, and wherein:

FIG. 1 is a perspective view of the medical illumination device within a sterile package and partially inserted into a shipping box;

FIG. 2 is a perspective view of the medical illumination device;

FIG. 3 is an exploded perspective view of the medical illumination device;

FIG. 4 is a partial section view of the medical illumination device providing further detail of the base;

FIG. 5 is a partial section view of the medical illumination device providing further detail of the light housing;

FIG. 6 is a partial sectional view of an alternate embodiment of the light housing;

FIG. 7 is a partial section view of the flexible body that supports the light housing; and

FIG. 8 is a schematic drawing of an embodiment of the electrical circuit for the light.

FIG. 9 is a perspective view of an embodiment of a medical illumination device having a plurality of flexible bodies and light sources;

### DESCRIPTION OF THE PREFERRED EMBODIMENT

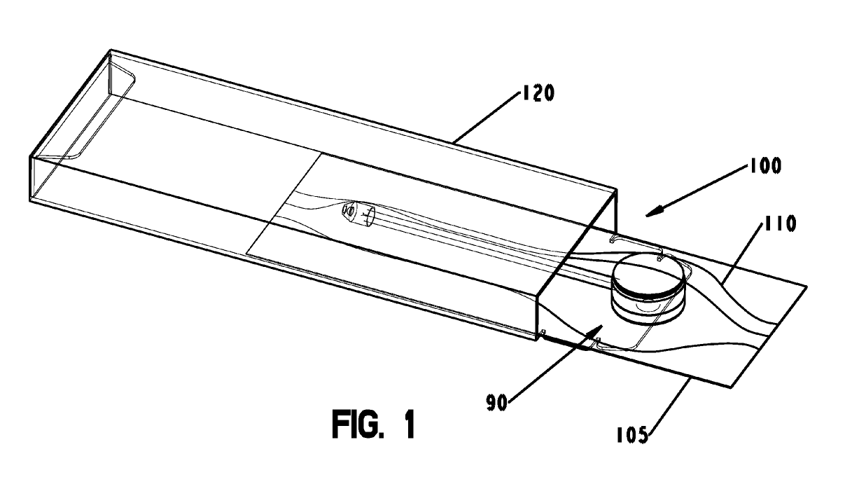

Generally, one embodiment of the system consists of a medical illumination device packaged within a sterile package. Referring to FIG. 1, medical illumination device 90, is shown packaged within sterile package 100, and partially inserted into a shipping box 120. Shipping box 120 is used for shipping and storage of the medical illumination device 90 in the sterile condition. Sterile package 100 consists of a back 105 and a cover 110, that are sealed to one another, completely surrounding medical illumination device 90. When medical illumination device 90 is sterilized, as long as sterile package 100 is not opened, the device will remain sterile until use of the device is desired. Back 105 and cover 110 may consist of Tyvek ®, foil, plastic, or other coated paper or polymer that maintains a barrier impervious to contaminants and maintains sterility, such as a sterility assurance level of 1 ×10⁻⁶. When illumination device 90 is desired for use, the user removes package 100 from box 120, opens back 105 and cover 110, and removes illumination device 90 in the sterile condition.

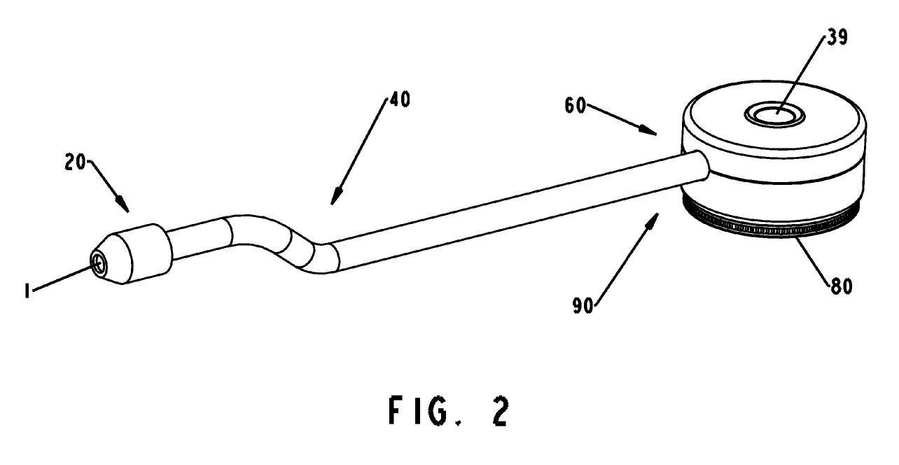

Referring to FIG. 2, medical illumination device 90 includes a light source 1, a light housing 20, a push-button 39, a flexible body 40, a base 60, and an attachment component 80.

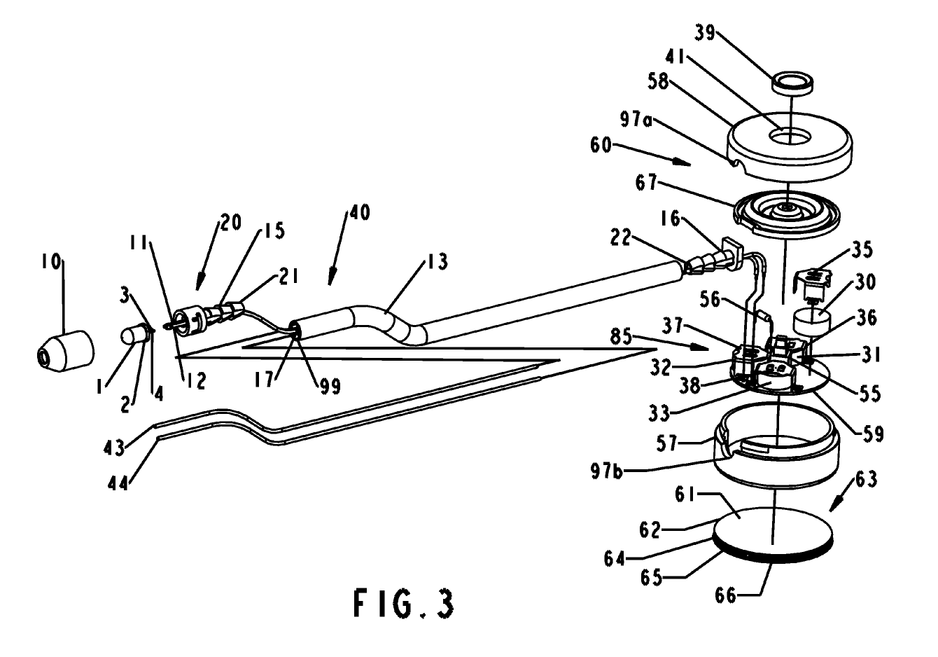

Referring to FIG. 3, an exploded view of the device provides a more detailed depiction of the internal and external components of one embodiment of illumination device 90. In this figure, the components of light source 1 and light housing 20 are shown. The light housing 20 includes a light holder 15, light source 1 and a light housing sleeve 10. Since the device may be used in body fluids, wound irrigation solutions or other liquids, light housing sleeve 10 may be made of a material and design that seals around light source 1 and light holder 15. In this way fluid would not contaminate or possibly disrupt the operation of the light and the light would not negatively impact the surgical procedure. Light housing 10 sealably encloses light source 1 and light holder 15 by sealably attaching to flexible tube 13. Alternatively, the device may not be sealed or only partially sealed. The device may be used as a single use device and simply disposed after use.

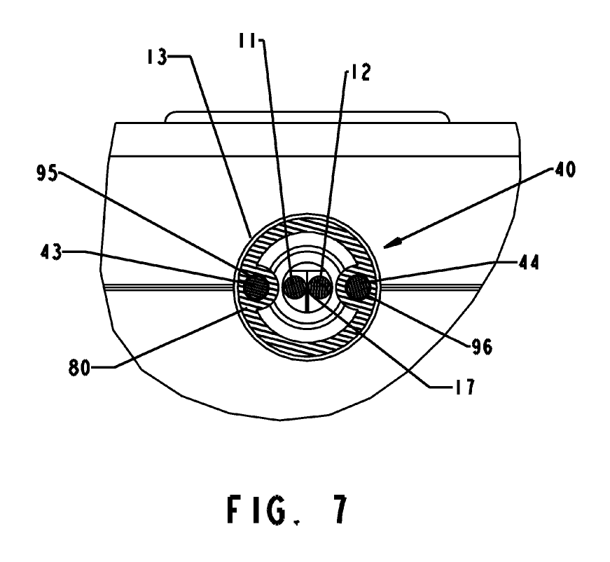

In FIG. 3, flexible body means or flexible body 40 includes flexible tube 13 and bendable shafts 43 and 44 positioned inside flexible body 40. Flexible body 40 is also shown in FIG. 7 and is described in more detail below. Light housing 20 is attached to flexible body 40 by inserting barbed end 21 of light holder 15 into the distal end 99 of flexible tube 13. Light holder 15 in this embodiment holds lighting means or light source 1 (such as an LED, or other electrically powered lighting) by snapping a collar feature 2 of light source 1 into light holder 15. Light holder 15 is pressed into flexible tube 13 and light housing sleeve 10 is pressed over light holder 15. Light source 1 in this embodiment has two voltage leads 3 and 4, which are electrically connected, such as by soldering, to conductors (insulated wires) 11 and 12, respectively. Conductors 11 and 12 are positioned inside flexible tube 13 via conductor channel 17 of flexible tube 13 and electrically connected, such as by soldering, to printed circuit (PC) board 59 of base 60. Flexible tube 13 is inserted over the barbed end 22 of tube support 16 to secure flexible body 40 to base 60.

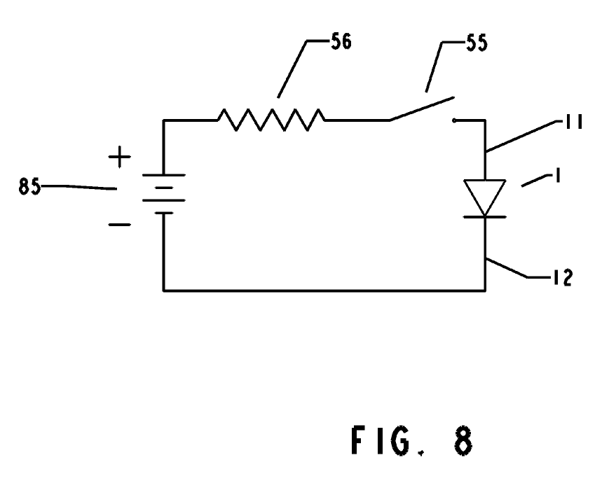

Light source 1, is powered by a power means or power source 85, including batteries, such as four button cell batteries 30, 31, 32 and 33, secured and maintained in electrical contact to PC board 59 with metal battery clips 35, 36, 37 and 38, respectively. Push-button 39, which is attached to a switch 55, such as a latching switch, activates the electrical circuit that powers the device. Switch 55 is electrically connected to PC board 59. Switch 55, alternates between an “off” position and an “on” position, thereby current is provided from batteries 30, 31, 32, 33 of power source 85 to light source 1 via conductors 11 and 12, which are also electrically connected to PC board 59. The voltage of the circuit is regulated by a resistor 56, which is in series with the circuit. A schematic of the electrical circuit is shown in FIG. 8. Light 1 remains in the lighted state until push-button 39 is pressed, causing switch 55 to transfer from the “on” position to the “off” position.

The PC board 59 and attached components are secured within base 60 which includes a base shell 57 and a cover 58. A seal 67, which is preferably flexible, is positioned over switch 55 to provide a moisture barrier. Seal 67 is positioned into the base shell 57, and forms a seal with the base to protect PC board 59 from excessive moisture so that a wet surgical environment may not disrupt operation of the light.

Base shell 57 and cover 58 secure or snap together, securing a tube support 16 in mating cutouts 97a, 97b to base 60. Push button 39 is positioned in button opening 41 in cover 58. Push-button 39 activates switch 55, under seal 67 within the base 60.

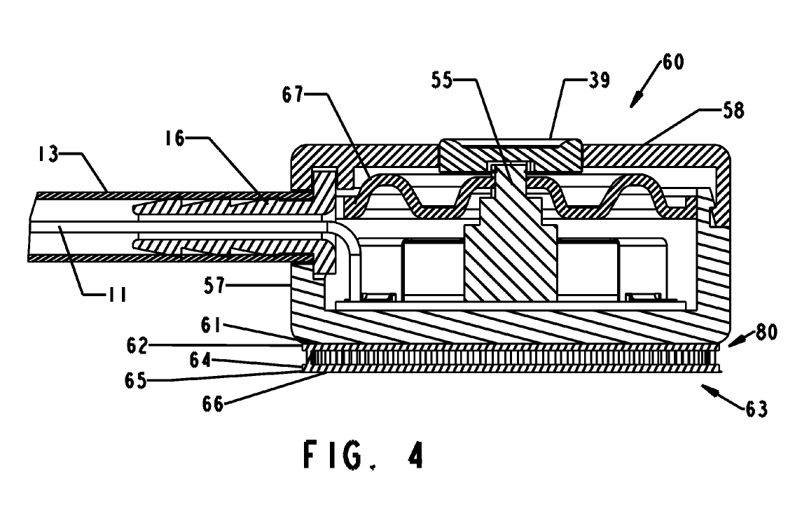

Attachment component 80 is attached to the bottom of base 60. In this embodiment, attachment component 80 is a round hook and loop (Velcro®) pad 63 attached to base 60. Referring to FIG. 4, Attachment component 80 includes hook and loop pad 63. Hook and loop pad 63 includes base adhesive 61, hook pad 62, loop pad 64, mounting adhesive 65, and cover sheet 66. Base adhesive 61 and mounting adhesive 65 may be any type of suitable adhesive such as pressure sensitive adhesive. Base 60 has sufficient surface area contacting the attachment point to effectively provide stability for repositioning of flexible body 40. Mounting adhesive 65 has sufficient surface holding strength to the attachment point to effectively provide stability for repositioning of flexible body 40. The base 60 may have a surface area of at least 1.0 square inch and comprises at least 50%, preferably at least 60%, of the weight of the illumination device 90. The combination of the base surface area and base weight percentage of the base 60 and the attachment component 80 holding strength effectively provide stability for repositioning of the flexible body 40 and/or securing of the device for use.

Attachment component 80 may include mounting adhesive 65 that is exposable upon removal of attachment component 80 from the sterile packager 100. Alternatively, attachment component 80 may also include a coversheet 66 that is attached to sterile package 100 so that removal of coversheet 66 is facilitated upon removal of attachment component 80 from sterile package 100. Alternatively, coversheet 66 may be integral with sterile package 100. Attachment component 80 may include a first portion, such as loop pad 64, and a second portion, such as hook pad 62, such that the first portion is attachable to an attachment point and the second portion is repositionable relative to the first portion without detaching the first portion from the attachment point. Alternatively, the first portion and the second portion may be separately packaged and separately removable from sterile package 100. Separately packaging the first portion from the second portion allows the practictioner to attach the first portion to an acceptable area, and then later attach and reposition the second portion as needed.

The base 60 is mounted in position by, first, peeling adhesive coversheet 66 from loop pad 64 of hook and loop pad 63, thus exposing mounting adhesive 65. With mounting adhesive 65 thus exposed, the user secures base 60 to a surface near where illumination is required by pressing it in place. Base 60 is easily repositioned by separating hook pad 64 from loop pad 62, rotating the pads 62, 64 relative to each other and reattaching pads 62, 64 to each other, thereby rotating base 60 relative to the surface to which it had been mounted.

Alternate embodiments of attachment component 80 or attachment means include an alligator clip, a hook, a clamp, adhesive, tape, staples, or other attachment means or components that are capable attaching base 60 to the patient or other local attachment points such as a surgical drape, the patient, or patient's clothes in the case of an emergency situation.

FIG. 4 is a cross-section of the device, providing further detail of base 60.

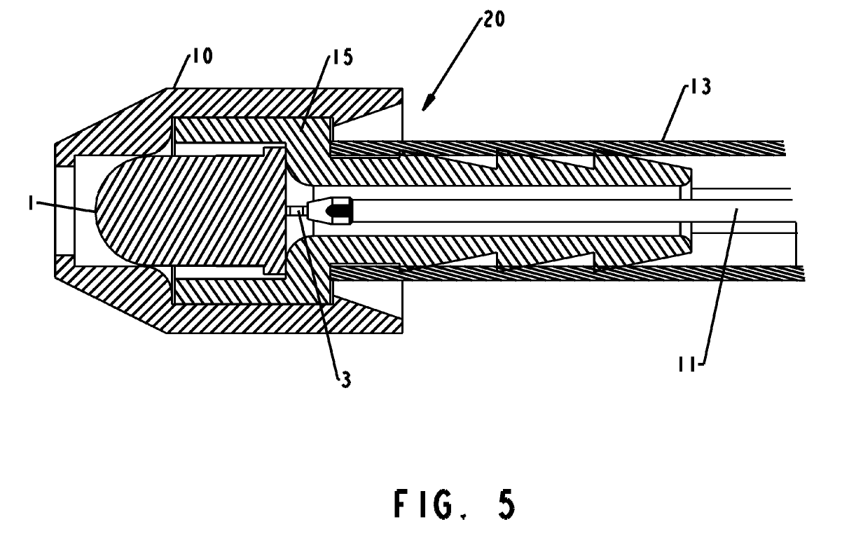

FIG. 5 is a cross-section of the device, providing further detail of light housing 20.

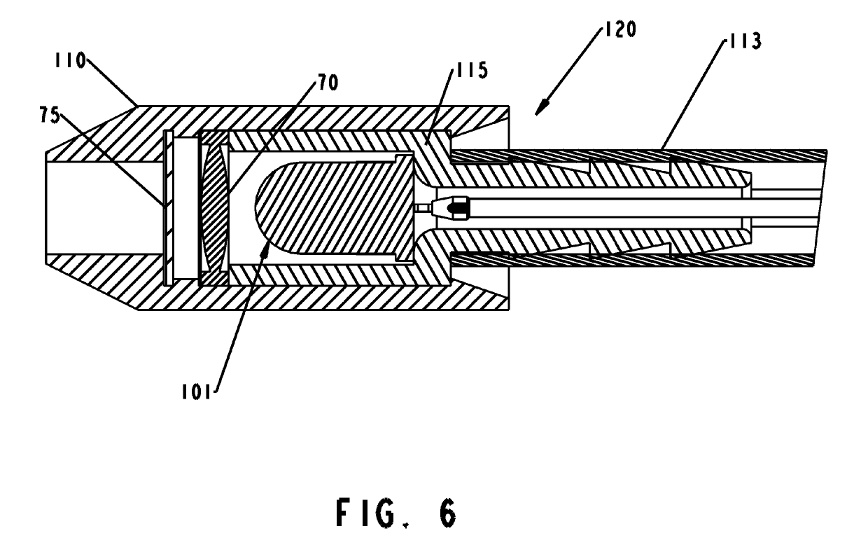

FIG. 6 is partial sectional view of an alternate embodiment similar to light housing 20 described above. Light housing 1 20 includes light housing sleeve 110, light source 101, light holder 115, and flexible tube 113. This alternative embodiment includes a lens 70 positioned in the end of the light housing sleeve 110 to provide a narrow or wide light beam, depending on the type of lens used. In addition, this embodiment may have a filter 75, such as a color or polarizing filter, positioned in the end of light housing sleeve 110 to provide filtered light to improve visibility of specific features of the body. Both the lens and the filter may be held in place by frictional fit, whereby they are snapped into position. This embodiment may include both lens 70 and filter 75 snapped or positioned into light housing sleeve 110.

Furthermore, a multitude of lenses and/or filters and/or polarizers may be used individually or in combination to provide specific types of light desired by the user. The lenses may also be orientated within light housing 120 to be adjustable and provide adjustment to the light from a narrow light beam to a wide light beam by the user. Since the device may be used in body fluids, wound irrigation solutions or other liquids, light housing sleeve 110 may be made of a material and design that seals around filter 75 and lens 70 and light holder 115. In this way fluid would not contaminate or possibly disrupt the operation of the light and the light would not negatively impact the surgical procedure. Housing sleeve 110 sealably encloses light source 101, filter 75, lens 70, and light housing 115 by sealably attaching to flexible tube 113. Alternatively, the device may not be sealed or only partially sealed. The device may be used as a single use device and simply disposed after use.

FIG. 7 shows a cross-section of the medical illumination device, providing further detail of flexible body 40. Inside flexible tube 13 are two shaft channels 95, 96 on opposite sides of flexible tube 13 within a tube wall 80 of flexible body 40. Within these two small shaft channels are bendable shafts 43 and 44 that hold the shape into which they have been bent and provide a flexible self-sustaining shape to the bendable shafts and to flexible tube 13. When flexible tube 13 is bent into the desired shape to orient the light source 1 at the proper angle for a medical procedure, bendable shafts 43 and 44 bend within the tube wall 80 and tend to hold flexible tube 13 in the desired shape or position. In the center portion of the tube is channel 17 that encloses conductors 11 and 12 between light source 1 and power source 85. The design provides for a hollow, bendable, flexible, self-sustaining tube that can be bent without putting excessive stress on the conductors for the circuit.

FIG. 8 is a schematic drawing depicting the electrical circuit of illumination device 90. Power source 85 provides current to light source 1 when latching switch 55 is put into the “on” position (when the switch is closed). Resistor 56 regulates the current so that light source 1 receives the proper amount of power. Conductors 11 and 12 provide power to light source 1 from power source 85.

The above described circuit shown in FIG. 8, may also have alternate embodiments, for example, switch 55 may be a membrane switch, snap-dome switch, levered switch or similar devices. Additional supporting circuitry to utilize alternative switch embodiments may also be implemented to provide the necessary functionality.

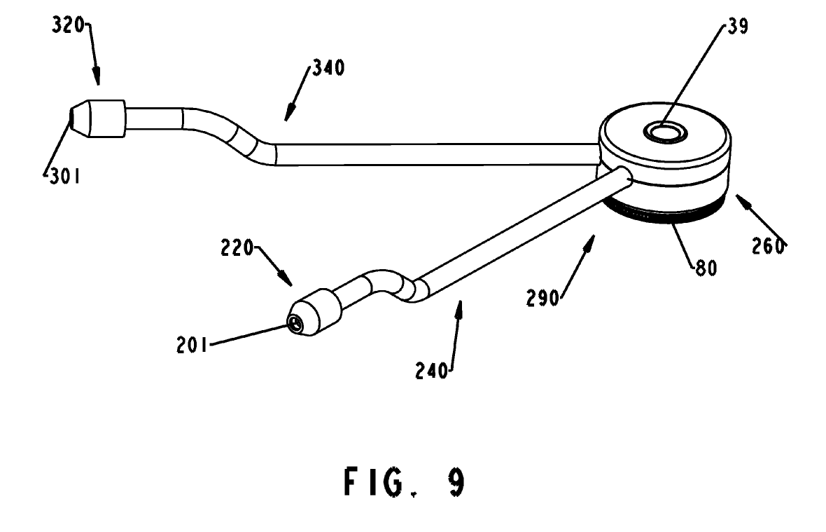

Referring to FIG. 9, alternate embodiment medical illumination device 290 includes first light source 201 and second light source 301, first light housing 220 and second light housing 320, a push-button 39, first flexible body 240 and second flexible body 340, a base 260, and an attachment component 80. Device 290 may have a plurality of light sources and flexible bodies attaching the light sources to the base. Additional light sources and flexible bodies provide the ability to direct light from multiple directions at the same time.

While there is shown and described the present preferred embodiment of the invention, it is understood that this invention is not limited thereto but may be variously embodied to practice within the scope of the following claims. From the foregoing description, it will be apparent that various changes may be made without departing from the spirit and scope of the invention as defined by the following claims. It is to be understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

What is claimed is:

1. A medical illumination device comprising:
a base containing a power source, said base further comprising an attachment component, said attachment component attachable to an attachment point;
a light housing enclosing a light source;
a flexible body attached to said base and said light housing, said light source electrically connected to said power source via conductors enclosed in said flexible body, said flexible body having a self-sustaining shape; and
wherein said base provides stability for repositioning of said flexible body.

2. The device of claim 1 wherein said base has sufficient surface area contacting the attachment point to effectively provide stability for repositioning of said flexible body.

3. The device of claim 2 wherein said base has a surface area of at least 1.0 square inch.

4. The device of claim 1 wherein said base comprises at least 60% of the weight of the device.

5. The device of claim 3 wherein said base comprises at least 50% of the weight of the device.

6. The device of claim 1 wherein said base has sufficient surface area contacting the attachment point, sufficient base weight, and sufficient holding strength to effectively provide stability for repositioning of said flexible body.

7. The device of claim 6 wherein said base comprises at least 50% of the weight of the device and said base has a surface area of at least 1.0 square inch.

8. The device of claim 1 wherein said flexible body comprises at least one bendable shaft positioned inside said flexible body.

9. The device of claim 1 wherein said light housing further comprises a lens for adjusting of a light beam.

10. The device of claim 1 wherein said light housing further comprises a filter for improving visibility.

11. The device of claim 1 wherein said flexible body is sealably attached to said base and said light housing sealably encloses said light source.

12. The device of claim 1 further comprising a second light housing enclosing a second light source and a second flexible body attaching said second light housing to said base.

13. The device of claim 1 wherein said attachment component comprises a first portion and a second portion, wherein said first portion is attachable to the attachment point and said second portion is repositionable relative to said first portion without detaching said first portion from the attachment point.

14. A medical illumination device comprising:
a base containing a power source, said base further comprising an attachment component;
a light housing enclosing a light source;
a flexible body attached to said base and said light housing, said light source electrically connected to said power source via conductors enclosed in said flexible body, said flexible body having a self-sustaining shape; and
said attachment component comprising a first portion and a second portion, wherein said first portion is attachable to an attachment point and said second portion is repositionable relative to said first portion without detaching said first portion from the attachment point.

15. The device of claim 14 wherein said attachment component comprises a hook and loop pad.

16. The device of claim 14 wherein said flexible body comprises at least one bendable shaft positioned inside said flexible body.

17. The device of claim 14 wherein said light housing further comprises a lens for adjusting of a light beam.

18. The device of claim 14 wherein said light housing further comprises a filter for improving visibility.

19. The device of claim 14 wherein said flexible body is sealably attached to said base and said light housing sealably encloses said light source.

20. The device of claim 14 wherein said base has sufficient surface area contacting the attachment point, sufficient base weight, and sufficient holding strength to effectively provide stability for repositioning of said flexible body.

21. The device of claim 19 wherein said base comprises at least 50% of the weight of the device and said base has a surface area of at least 1.0 square inch.

22. The device of claim 14 further comprising a second light housing enclosing a second light source and a second flexible body attaching said second light housing to said base.

23. A medical illumination device system comprising:
a base containing a power source, said base further comprising an attachment component, said attachment component further comprising a first portion and a second portion, said first portion attachable to an attachment point and said second portion repositionable relative to said first portion without detaching said first portion from the attachment point;
a light housing enclosing a light source;
a flexible body attached to said base and said light housing, said light source electrically connected to said power source via conductors enclosed in said flexible body, said flexible body having a self-sustaining shape, said flexible body comprising at least one bendable shaft positioned inside said flexible body; and
wherein said base provides stability for repositioning of said flexible body and wherein said base has sufficient surface area contacting the attachment point, sufficient base weight, and sufficient holding strength to effectively provide stability for repositioning of said flexible body, and wherein said base comprises at least 60% of the weight of the device and said base has a surface area of at least 1.0 square inch.

24. The device of claim 23 further comprising a second light housing enclosing a second light source and a second flexible body attaching said second light housing to said base.

25. The device of claim 23 wherein said light housing further comprises a lens and a filter.

26. The device of claim 23 wherein said flexible body is sealably attached to said base and said light housing sealably encloses said light source.
